(19) United States
(12) Patent Application Publication
(10) Pub. No.: US 2025/0127564 A1
(43) Pub. Date: Apr. 24, 2025

(54) DEFLECTABLE SHEATH WITH INDICATOR

(71) Applicant: Boston Scientific Medical Device Limited, Galway (IE)

(72) Inventor: Jan-Hung Chen, St. Paul, MN (US)

(21) Appl. No.: 18/917,729

(22) Filed: Oct. 16, 2024

Related U.S. Application Data
(60) Provisional application No. 63/591,497, filed on Oct. 19, 2023.

(51) Int. Cl. A61B 18/14; A61B 18/00; A61B 90/00

(52) U.S. Cl. CPC A61B 18/1492; A61B 2018/00172; A61B 2018/00184; A61B 2018/00351; A61B 2018/00577; A61B 2018/0091; A61B 2018/1475; A61B 2090/0807; A61B 2090/0811

(57) ABSTRACT

A steerable sheath is disclosed. The steerable sheath includes an elongate member, the elongate member having a proximal portion and a distal portion terminating in a deflectable distal tip. The steerable sheath further includes a handle having a handle proximal portion and a handle distal portion that is connected to the proximal portion of the elongate member. The handle of the steerable sheath includes a knob positioned at the distal portion of the handle; a slider running through a portion of the handle having a raised thread and an inverted thread; and a position ring located proximate a position indicator, wherein rotation of the knob acts upon the raised thread to cause a linear movement of the slider which acts upon the inverted thread to cause a rotational movement of the position ring.

### CROSS REFERENCE TO RELATED APPLICATIONS

The present application claims priority to U.S. Provisional Application No. 63/591,497, filed Oct. 19, 2023, the entire disclosure of which is incorporated herein by reference.

### TECHNICAL FIELD

The present invention relates generally to systems and methods for deflectable medical catheters. More specifically, the present invention is concerned with a steerable sheath having a deflectable distal tip curve position indicator which provides a visual cue of the steerable sheath deflectable distal tip curve direction.

### BACKGROUND

Existing steerable sheaths, like the SureFlex steerable sheath, do not indicate on the handle, or anywhere else on the device, the amount and/or the direction of deflection of the distal portion of the sheath. Generally, for steerable sheaths, deflection is possible based on rotation of a knob on the sheath. If more than one rotation of the knob is required to fully deflect the sheath, it is difficult to correlate the amount of knob rotation with the amount of distal portion sheath deflection. Additionally, when the sheath is inserted in the body of a patient, the user must rely on fluoroscopy to indicate the distal tip position of the sheath. This process increases radiation exposure to both the user and the patient. Moreover, if multiple steerable sheaths are used simultaneously, the sheaths cannot be distinguished based on their curve positions. This may especially create an array of issues during procedures which require fine adjustment of the position of the distal tip, like in transseptal crossing procedures and ablating tissue within the heart of a patient.

Against this background, there exists a continuing need in the industry to provide improved steerable sheaths having a sheath curve position indicator. Specifically, for use in transseptal procedures. An object of the present invention is therefore to provide such a steerable sheath.

### SUMMARY

In Example 1, a steerable sheath includes an elongate member defining a lumen, the elongate member having a proximal portion and a distal portion terminating in a deflectable distal tip. The steerable sheath further includes a handle defining a handle lumen having a handle proximal portion and a handle distal portion that is connected to the proximal portion of the elongate member, the handle comprising: a rotatable knob; a slider running through a portion of the handle having a raised thread and an inverted thread, a first direction of the raised thread being inverse to a second direction of the inverted thread; and a position ring located proximate a position indicator, the position ring and the position indicator each having one or more bevel gears connecting the position ring to the position indicator, the position indicator located proximate the knob, wherein rotation of the knob acts upon the raised thread to cause a linear movement of the slider which acts upon the inverted thread to cause a rotational movement of the position ring.

Example 2 is the steerable sheath of Example 1 wherein the one or more bevel gears of the position ring rotate the one or more bevel gears of the position indicator.

Example 3 is the steerable sheath of any of Examples 1-2 wherein the rotational movement of the position ring translates to rotation of the position indicator.

Example 4 is the steerable sheath of Example 1 wherein the direction of the position indicator coincides with the curved direction of the deflectable distal tip.

Example 5 is the steerable sheath of Example 1 wherein the handle further includes two or more pull wires connected to the slider.

Example 6 is the steerable sheath of Example 5 wherein the pull wires control the curve direction of the deflectable distal tip.

Example 7 is the steerable sheath of Example 1 wherein the position ring is connected to the inverted thread by a connector tab.

Example 8 is the steerable sheath of Example 1 wherein the knob is rotated by a user depending on the desired direction of curvature of the deflectable distal tip.

Example 9 is the steerable sheath of any of Examples 1-8 wherein the position indicator provides a visual cue to the user indicating the curve direction of the deflectable distal tip of the steerable sheath.

Example 10 is the steerable sheath of Example 1 wherein the pitch of the raised thread is higher than the pitch of the inverted thread.

Example 11 is the steerable sheath of Example 1 wherein the pitch of the raised thread is between 10 mm to 15 mm.

Example 12 is the steerable sheath of Example 1 wherein the pitch of the inverted thread is between 45 mm to 55 mm.

Example 13 is the steerable sheath of Example 1 wherein the system further includes a puncture device configured to advance through the steerable sheath.

Example 14 is the steerable sheath of Example 1 wherein the system further includes a medical therapy device configured to advance through the steerable sheath.

Example 15 is the steerable sheath of Example 14 wherein the medical therapy device is an ablation catheter.

In Example 16, a steerable sheath includes an elongate member defining a lumen, the elongate member having a proximal portion and a distal portion terminating in a deflectable distal tip. The steerable sheath further includes a handle defining a handle lumen having a handle proximal portion and a handle distal portion that is connected to the proximal portion of the elongate member, the handle comprising: a rotatable knob; a slider running through a portion of the handle having a raised thread and an inverted thread, a first direction of the raised thread being inverse to a second direction of the inverted thread; and a position ring located proximate a position indicator, the position ring and the position indicator each having one or more bevel gears connecting the position ring to the position indicator, the position indicator located proximate the knob, wherein rotation of the knob acts upon the raised thread to cause a linear movement of the slider which acts upon the inverted thread to cause a rotational movement of the position ring.

Example 17 is the steerable sheath of Example 16 wherein the one or more bevel gears of the position ring rotate the one or more bevel gears of the position indicator, and wherein the rotational movement of the position ring translates to rotation of the position indicator.

Example 18 is the steerable sheath of Example 16 wherein the direction of the position indicator coincides with the curved direction of the deflectable distal tip.

Example 19 is the steerable sheath of Example 16 wherein the handle further includes two pull wires connected to the slider, and wherein the pull wires control the curve direction of the deflectable distal tip.

Example 20 is the steerable sheath of Example 16 wherein the position ring is connected to the inverted thread by a connector tab.

Example 21 is the steerable sheath of Example 16 wherein the knob is rotated by a user depending on the desired direction of curvature of the deflectable distal tip, and wherein the position indicator provides a visual cue to the user indicating the curve direction of the deflectable distal tip of the steerable sheath.

Example 22 is the steerable sheath of Example 16 wherein the pitch of the raised thread is higher than the pitch of the inverted thread.

Example 23 is the steerable sheath of Example 16 wherein the pitch ratio of the raised thread to the inverted thread is 4:1.

Example 24 is the steerable sheath of Example 16 wherein the pitch of the raised thread is between 10 mm to 15 mm.

Example 25 is the steerable sheath of Example 16 wherein the pitch of the inverted thread is between 45 mm to 55 mm.

Example 26 is the steerable sheath of Example 16 wherein the system further includes a puncture device and an ablation device configured to advance through the steerable sheath.

In Example 27, a steerable sheath for transseptal crossing and subsequent therapy includes an elongate member defining a lumen, the elongate member having a proximal portion and a distal portion terminating in a deflectable distal tip. The steerable sheath further includes a handle defining a handle lumen having a handle proximal portion and a handle distal portion that is connected to the proximal portion of the elongate member. The handle further comprises a knob positioned at the distal portion of the handle; a slider running through a portion of the handle having a raised thread and an inverted thread, a first direction of the raised thread being inverse to a second direction of the inverted thread; and a position ring located proximate a position indicator, the position ring and the position indicator each having one or more bevel gears connecting the position ring to the position indicator, the position indicator located proximate the knob, wherein rotation of the knob acts upon the raised thread to cause a linear movement of the slider which acts upon the inverted thread to cause a rotational movement of position ring, and wherein the one or more bevel gears of the position ring rotate the one or more bevel gears of the position indicator, and wherein the rotational movement of the position ring translates to rotation of the position indicator.

Example 28 is the steerable sheath of Example 27 wherein the direction of the position indicator coincides with the curved direction of the deflectable distal tip.

Example 29 is the steerable sheath of Example 27 wherein the handle further includes two pull wires connected to the slider, and wherein the pull wires control the curve direction of the deflectable distal tip.

Example 30 is the steerable sheath of Example 27 wherein the position ring is connected to the inverted thread by a connector tab.

Example 31 is the steerable sheath of Example 27 wherein the knob is rotated by a user depending on the desired direction of curvature of the deflectable distal tip, and wherein the position indicator provides a visual cue to the user indicating the curve direction of the deflectable distal tip of the steerable sheath.

Example 32 is the steerable sheath of Example 27 wherein the pitch of the raised thread is higher than the pitch of the inverted thread.

Example 33 is the steerable sheath of Example 27 wherein the system further includes a puncture device and a subsequent medical therapy device configured to advance through the steerable sheath.

Example 34 is the steerable sheath of Example 33 wherein the system further includes a subsequent medical therapy device configured to advance through the steerable sheath.

In Example 35, a method of using a steerable sheath for transseptal crossing and subsequent therapy includes providing an elongate member defining a lumen, the elongate member having a proximal portion and a distal portion terminating in a deflectable distal tip. The method of using a steerable sheath further includes advancing a handle defining a handle lumen having a handle proximal portion and a handle distal portion that is connected to the proximal portion of the elongate member, the handle comprising: a knob positioned at the distal portion of the handle; a slider running through a portion of the handle having a raised thread and an inverted thread, a first direction of the raised thread being inverse to a second direction of the inverted thread; and a position ring located proximate a position indicator, the position ring and the position indicator each having one or more bevel gears connecting the position ring to the position indicator, the position indicator located proximate the knob, wherein rotation of the knob acts upon the raised thread to cause a linear movement of the slider which acts upon the inverted thread to cause a rotation movement position ring.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of a transseptal crossing system utilizing a steerable sheath for use with a dilator, according to embodiments of the present disclosure.

FIG. 2 is a schematic illustration of a portion of a catheter device for use in ablating heart tissue, according to embodiments of the present disclosure.

FIG. 3 is an illustration of the steerable sheath of FIG. 1 having a deflectable distal region, according to embodiments of the present disclosure.

FIGS. 4A-4B are sideview illustrations of the steerable sheath of FIG. 3 having a slider with two threads which run longitudinally through the steerable sheath, according to embodiments of the present disclosure.

FIGS. 5A-5C are schematic illustrations of the steerable sheath with a position indicator providing a visual cue of the direction of the steerable sheath distal portion deflection, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

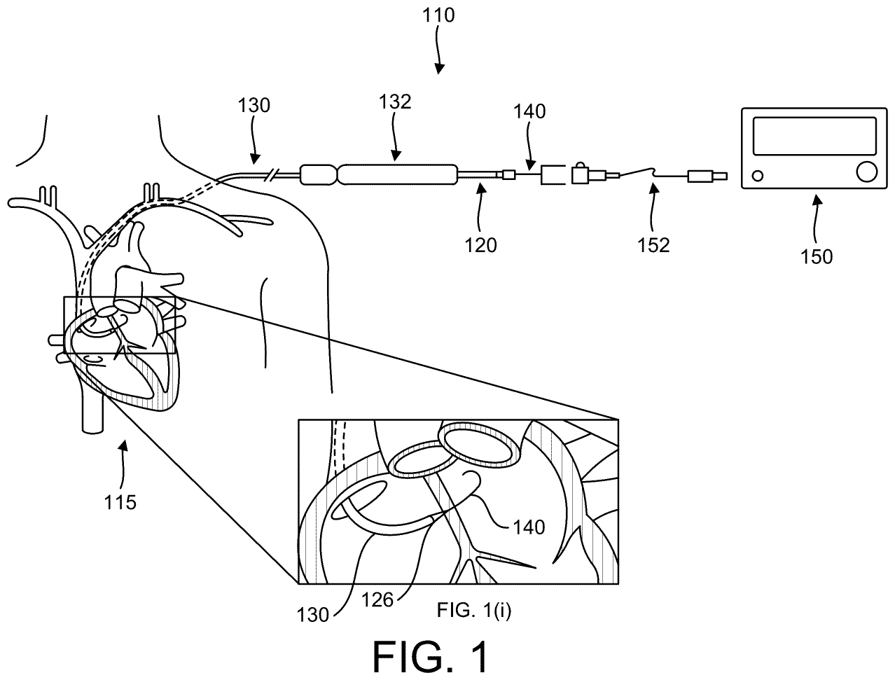

FIG. 1 is an illustration of a transseptal access system 110 that incorporates embodiments of devices of the present invention and that may be utilized during the course of an inventive procedure as described further herein. As illustrated, system 110 includes a steerable device such as a steerable sheath 130 with a dilator 120 inserted therein, and a guidewire 140 inserted into the dilator 120. The steerable sheath 130 and the dilator 120 each define a respective lumen through which devices may be inserted. Although a steerable sheath is discussed throughout this application, it will be evident to one of skill in the art that other steerable devices or articulating components may be used. For ease of explaining the fundamental principles of the invention, “steerable sheath” will be used throughout the specification as an example of a steerable or articulating device. Alternatively, in some embodiments, a fixed curve sheath may be utilized in place of an articulating sheath, depending on the access point and tissue puncture site chosen by a user.

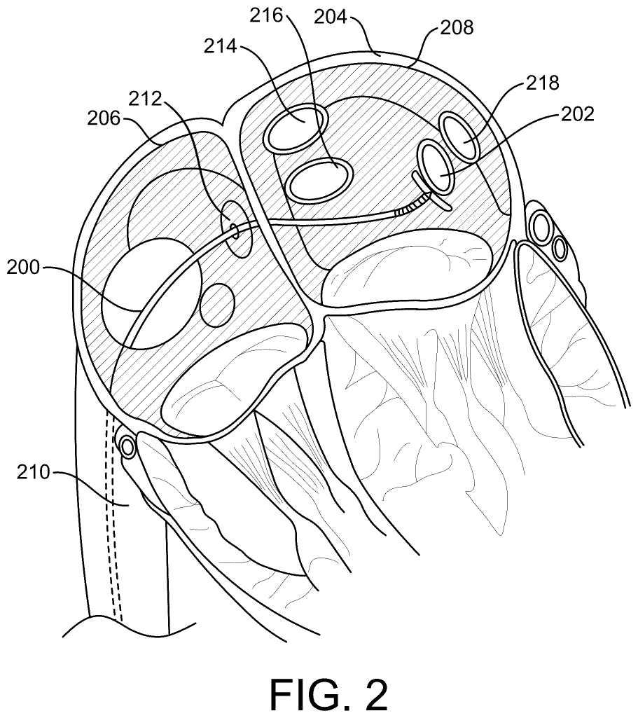

The guidewire 140 is connected to a generator 150 by a connector 152. The steerable sheath 130 includes a steerable sheath handle 132. In some embodiments, the steerable sheath is unidirectional, i.e., it allows deflection in a single direction. In other embodiments, a bi-directional sheath may be used. FIG. 2(i) depicts an expanded view of a portion of a heart 115 illustrating a distal portion of steerable sheath 130, a distal tip 126 of the dilator 120, and the guidewire 140. In some embodiments, the guidewire 140 may be a radiofrequency (RF) perforation device.

As is known, and shown in FIG. 1, the human heart 115 has four chambers, a right atrium, a left atrium, a right ventricle and a left ventricle. Separating the right atrium and the left atrium is an atrial septum, and separating the right ventricle and the left ventricle is a ventricular septum. As is further known, deoxygenated blood from the patient's body is returned to the right atrium via an inferior vena cava (IVC) or a superior vena cava (SVC).

Various medical procedures have been developed for diagnosing or treating physiological ailments originating within the left atrium and associated structures. Exemplary such procedures include, without limitation, deployment of diagnostic or mapping catheters within the left atrium for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium are merely illustrative and in no way limiting with respect to the present disclosure.

The medical procedure of system 110 is an exemplary embodiment for providing access to the left atrium using the transseptal access system with a steerable sheath 130 for subsequent deployment of the aforementioned diagnostic and/or therapeutic devices within the left atrium, as will be discussed in greater detail below. In some embodiments, a target site is accessed via the IVC, for example through the femoral vein, according to conventional catheterization techniques. In other embodiments, access to the target site on the atrial septum may be accomplished using a superior approach wherein the transseptal access system is advanced into the right atrium via the SVC.

In the assembled use state illustrated in FIG. 1, an RF perforation device can be disposed within the dilator 120, which itself can be disposed within the sheath 130. In one embodiment in which the transseptal access system 110 is deployed into the right atrium via the IVC, a user introduces a wire (not shown) into a femoral vein, typically the right femoral vein, and advances it towards the heart 115. The sheath 130 may then be introduced into the femoral vein over the wire, and advanced towards the heart 115. In one embodiment, the distal ends of the wire and sheath 130 are then positioned in the SVC. These steps may be performed with the aid of an imaging system, e.g., fluoroscopy or ultrasonic imaging. The dilator 120 may then be introduced into the sheath 130 and over the wire, and advanced through the sheath 130 into the SVC. Alternatively, the dilator 120 may be fully inserted into the sheath 130 prior to entering the body, and both may be advanced simultaneously towards the heart 115. When the wire, sheath and dilator 120 have been positioned in the SVC, the wire is removed from the body, and the sheath 130 and the dilator 120 are retracted so that their distal ends are positioned in the right atrium. The RF perforation device described can then be introduced into the dilator 120, and advanced toward the heart 115.

Subsequently, the user may position the distal end of the dilator 120 against the atrial septum, which can be done under imaging guidance. With the aid of the steerable sheath 130, the RF perforation device is then positioned such that the tip electrode is aligned with or protruding slightly from the distal end of the dilator 120. The dilator 120 and the RF perforation device may be dragged along the atrial septum and positioned, for example against the fossa ovalis of the atrial septum under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode at the desired target site. In addition, tactile feedback provided by medical RF perforation device is usable to facilitate positioning of the tip electrode at the desired target site.

With the tip electrode and dilator 120 positioned at the target site, energy is delivered from an energy source, e.g., an RF generator 150, through the RF perforation device to the tip electrode and the target site. In some embodiments, the energy is delivered at a power of at least about 5 W at a voltage of at least about 75 V (peak-to-peak), and functions to vaporize cells in the vicinity of the tip electrode, thereby creating a void or perforation through the tissue at the target site. The user then applies force to the RF perforation device so as to advance the tip electrode at least partially through the perforation. In these embodiments, when the tip electrode has passed through the target tissue, that is, when it has reached the left atrium, energy delivery is stopped. In some embodiments, the step of delivering energy occurs over a period of between about 1 second and about 5 seconds.

With the tip electrode of the RF perforation device having crossed the atrial septum, the dilator 120 can be advanced forward, with the tapered distal tip portion operating to gradually enlarge the perforation to permit advancement of the distal end of the sheath 130 into the left atrium.

In embodiments, the RF perforation device can be structurally configured to function as a delivery rail for deployment of a relatively larger bore therapy delivery sheath and associated dilator(s). In such embodiments, the dilator and the sheath 130 are withdrawn following deployment of the distal end portion of the RF perforation device into the left atrium. The anchoring function of the pre-formed distal end portion inhibits unintended retraction of the distal end portion, and corresponding loss of access to the perforated site on the atrial septum, during such withdrawal.

FIG. 2 is a schematic diagram illustrating a portion of a catheter device 200 positioned proximate a pulmonary vein 202 of a heart 204. As is known, the heart 204 includes a right atrium 206, a left atrium 208, the pulmonary vein 202, an inferior vena cava 210 and an intra-atrial septum 212. In various embodiments, the catheter device 200 is configured for use in ablating heart tissues to treat cardiac arrhythmias. In one embodiment, the catheter device 200 is configured for treating atrial fibrillation by ablating tissue surrounding the ostia of pulmonary veins 202 of the heart 204 and ensuring an electrical isolation of the pulmonary vein 202. In some embodiments, as discussed in greater detail below, the catheter device 200 may be a steerable catheter device for providing safe access to tissue within the heart 204 when performing ablation procedures and allowing deflection of the distal tip portion of the catheter 200 for better manipulability.

In the embodiment shown in FIG. 2, to position the catheter device 200 at a target tissue location in the pulmonary vein 202, the catheter device 200 can be inserted through a transseptal puncture, as explained in FIG. 1. The transseptal puncture permits a direct route from the right atrium 206 to the left atrium 208 via the intra-atrial septum 212 (by puncturing the septum 212 proximate or at the fossa ovalis). Once the catheter device 200 reaches the target tissue location in the pulmonary vein 202 in the left atrium 208 of the heart 204, the catheter device 200 is directed towards the pulmonary vein 202. The catheter device 200 can then be used to deliver radiofrequency (RF) energy to ablate the target tissues thereby isolating the pulmonary vein 202 from the rest of the heart 204 and preventing any pulses from the vein from getting into the heart 204. As shown, the left atrium 208 of the heart 204 includes three additional pulmonary veins 214, 216, and 218 that can be ablated as per the requirements in a manner similar to the ablation of the pulmonary veins 202.

The present disclosure describes novel devices and methods for a steerable sheath for transseptal crossing and subsequent advancement of therapy systems. As will be explained in greater detail herein, the embodiments of the present disclosure simplify the means of determining the deflection of the steerable sheath at a distal portion of the sheath, while providing enhanced manipulability to the user.

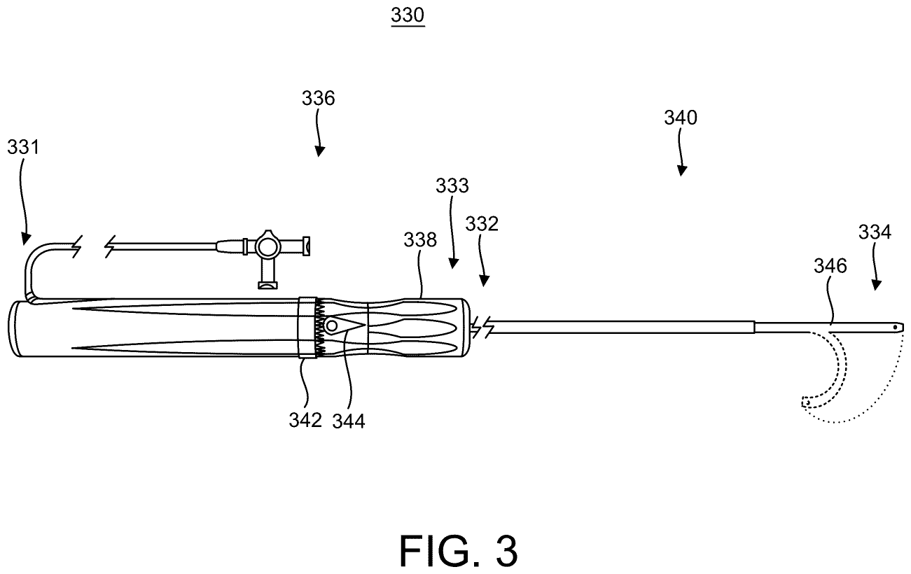

FIG. 3 is an illustration of a steerable sheath 330 comprising a deflectable distal tip 346 that is amenable to deflection upon actuation of a steerable actuation mechanism to access a region of tissue within a patient's heart, according to an embodiment of the present disclosure. As shown, the steerable sheath 330 comprises an elongate member 340 defining a lumen, the elongate member 340 having a proximal portion 332 and a distal portion 334, the distal portion 334 terminating in the deflectable distal tip 346. The steerable sheath 330 further includes a handle 336 defining a handle lumen, having a handle proximal portion 331 and a handle distal portion 333 that is connected to the proximal portion 332 of the elongate member 340. The handle further includes a knob 338 at the distal portion 333 of the handle 336, for user manipulation.

The handle 336 further includes a slider (not shown) running through an inside portion of the handle 336 having a raised thread and an inverted thread, wherein a first direction of the raised thread is inverse to a second direction of the inverted thread. Furthermore, the handle 336 includes a position ring 342 located proximate a position indicator 334, the position ring 342 and the position indicator 344 each having one or more bevel greats connecting the position ring 342 to the position indicator 344. In an embodiment, the position indicator 344 is located proximate the knob 338. In certain embodiments, and as will be discussed further herein, the bevel gears of the position ring 342 rotate the bevel gears of the position indicator 344, and thus the position indicator 344, clockwise or counterclockwise to signify the curvature of the deflectable distal region 340. In some embodiments, the steerable actuation mechanism includes the rotation of the knob 338 by a user. In some embodiments, as shown in FIG. 3, the deflectable distal tip 346 of the steerable sheath 330 has a range of deflection angles and can achieve a range of curvatures upon actuation.

In some embodiments, during use, a dilator may be inserted within the steerable sheath 330 for use therewith. In some embodiments, the dilator may include a flexible intermediate region that corresponds to the deflectable distal region 340 of the steerable sheath 330. In certain embodiments, this enables the steerable sheath 330 to reach its allowable range of curvatures or deflection upon actuation as minimal resistance is introduced by the dilator. In some embodiments, this enables the steerable sheath 330 to position the distal end region of the dilator at a desired target location within a region of tissue such as at a desired puncture location or site to enable the distal end region of the dilator to subsequently advance through to, for example, dilate the puncture site. In some embodiments, a puncture wire is configured to advance through the steerable sheath 330. In other further embodiments, a medical therapy device may be configured to advance through the steerable sheath 330. In still other embodiments, the medical therapy device is an ablation catheter.

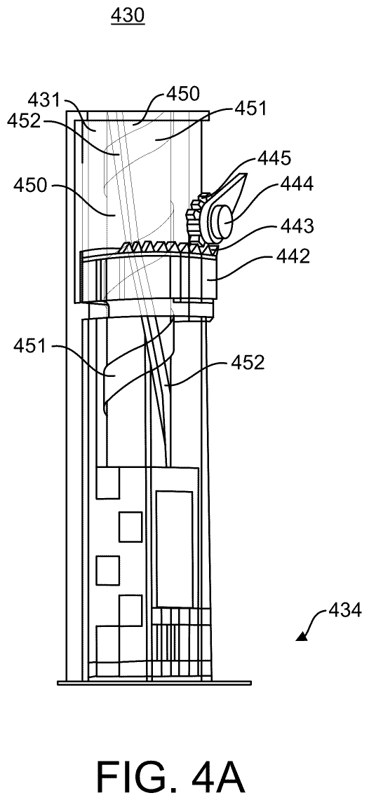

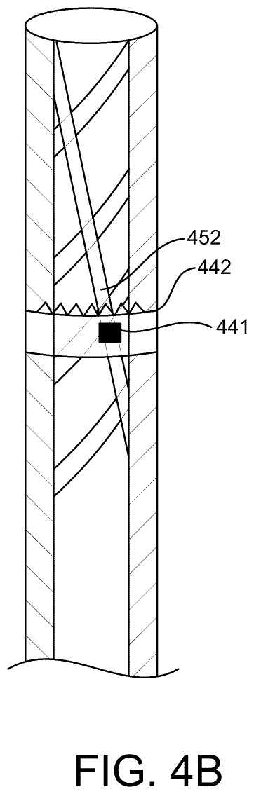

FIGS. 4A-4B are sideview illustrations of the steerable sheath of FIG. 3 having a slider 450 extending longitudinally through the steerable sheath 430 having two threads, according to embodiments of the present disclosure. The steerable sheath 430 may be substantially structurally and functionally identical to the steerable sheath 330 of FIG. 3, except as described in connection with FIGS. 4A-4B. As shown in FIG. 4A, the slider 450 extends longitudinally through a portion of a lumen 431 of the steerable sheath 430. As will be explained in further detail below, in some embodiments, the slider 450 fits into and is connected to the knob 338 of FIG. 3. The slider 450 includes a raised thread 451 and an inverted thread 452 carved thereon. As further shown in FIG. 4A, the steerable sheath also includes a position ring 442, having position ring bevel gears 443, located closer to the proximal portion 434 of the steerable sheath 430 than a position indicator 444, having position indicator bevel gears 445. In some embodiments, the position indicator bevel gears 445 are placed around the position indicator 444, as shown, in order to rotate the position indicator 444 clockwise or counterclockwise. As shown in FIG. 4B, the position ring 442 of the steerable sheath 430 further includes a connector tab 441 which connects the inverted thread 452 of the slider 450 with the position ring 442. Thus, the position indicator 444 is mated with the movements of the inverted thread 452.

Additionally, as shown, the direction of the raised thread 451 and the direction of the inverted thread 452 are inverse of each other. Furthermore, while the raised thread 451 includes a positive thread that protrudes from the slider 450 as it rotates around the slider 450, the inverted thread 452 has a negative thread that cuts into the slider 450 as it rotates around the slider 450. In some embodiments, the raised thread 451 and the inverted thread 452 have different thread pitches. In certain embodiments, the raised thread 451 has a lower pitch than the inverted thread 452. Both the raised thread 451 and the inverted thread 452 are spiral in nature. In certain embodiments, the pitch ratio of the raised thread 451 to the inverted thread 452 is 4:1. Thus, in other words, the raised thread 451 rotates around the slider 450 four times as the inverted thread 452 completes one rotation around the slider 450.

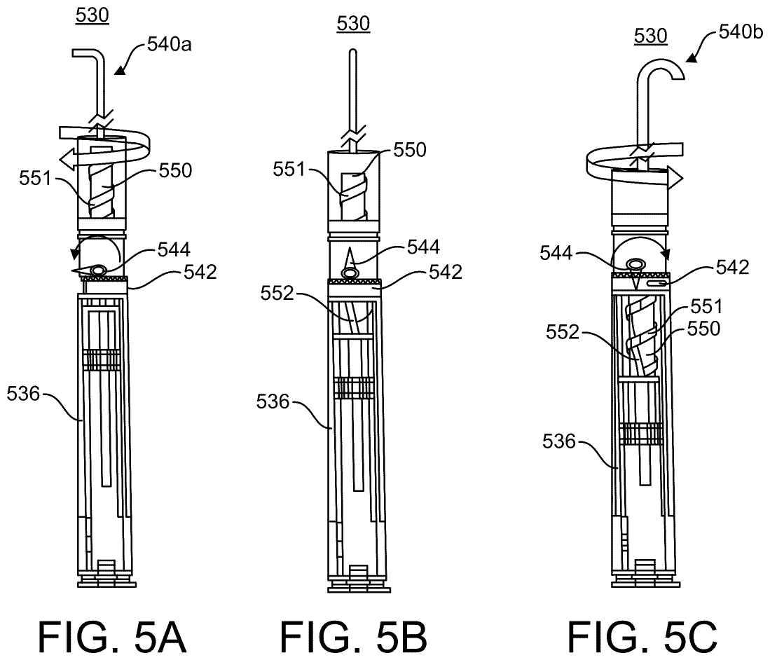

FIGS. 5A-5C are schematic illustrations of a steerable sheath 530 with a position indicator 544 providing a visual cue of the direction of a deflectable distal region 540a, 540b of the deflectable distal tip of FIG. 2, according to embodiments of the present disclosure. As illustrated in FIGS. 5A-5C, the steerable sheath 530 includes a handle 536 in which a slider 550 extends longitudinally through the lumen (not shown) of the handle 536 of the steerable sheath 530. In some embodiments, the slider 550 is double threaded. The slider 550 includes a raised thread 551 that protrudes from the slider 530 and an inverted thread 552 that is carved into the slider 550. The steerable sheath 530 further includes a position ring 542 that is connected to the inverted thread 542 via a connector tab and/or protrusion (not shown) of the steerable sheath 530. Additionally, as shown in FIG. 4A, the position ring 442 includes bevel gears that come in contact with the bevel gears of the position indicator 544. In some embodiments, the bevel gears of the position ring 542 rotate the bevel gears of the position indicator 544, and thus the position indicator 544, clockwise or counterclockwise to signify the curvature of the deflectable distal region 540a, 540b.

In some embodiments, the slider 550 fits into and is connected to the knob 338, as depicted in FIG. 3. In some embodiments, in use, a user may rotate the knob clockwise or counterclockwise depending on the desired direction of curvature of the deflectable distal region. In some embodiments, the raised thread 551 translates the rotation of the knob to a linear movement (i.e., up and down) of the slider 550. In some embodiments, the slider 550 includes two pull wires (not shown) that control the deflection angle of the deflectable distal region, for example 540a and 540b, of the steerable sheath 530. Thus, in one embodiment, as shown in FIG. 5A, when the knob is rotated clockwise by the user, the slider 540 will move up which in turn, because of the pull wires within the slider 540, will rotate the deflectable distal region 540a to the left. In another embodiment, as illustrated in FIG. 5C, when the knob is rotated counterclockwise, the slider will move down into the lumen of the steerable sheath 530 and because of the pull wires within the slider 540 the deflectable distal region 540b will turn to the right.

Furthermore, in a specific example, the rotation of the knob 338 is converted into a deflection of the steerable sheath 530 via a slide assembly (not shown). Generally, in some embodiments, the knob is rotatably coupled to a housing within the handle 536. In certain embodiments, the knob is co-operatively engaged with the slide assembly which is housed within a lumen defined by the housing of the handle 536. In a specific example, the knob is threadedly (i.e., the raised thread 551 and the inverted thread 552) engaged with the slide assembly. In one embodiment, the rotation of the knob causes a corresponding linear translation of the slide assembly within the lumen of the housing. This translation of the slide assembly is converted into a tensioning of the pair of pull wires coupled to the slide assembly and thereby resulting in a deflection of the sheath 530.

More specifically, in certain embodiments, the slider assembly is coupled to respective proximal ends of the pull wires that extend substantially along the length of the steerable sheath 530. A distal end (not shown) of each of the pull wires is coupled to a distal portion of the steerable sheath 530. In some embodiments, the rotation of the knob in one direction causes the slide assembly to translate proximally within the housing of the handle 536 pulling one of the pull wires to deflect the sheath 530 in a first direction, whereas the rotation of the knob in an opposing direction causes the slide assembly to translate distally within the housing pulling the other of the pull wires to deflect the sheath 530 in a second direction. In one example, in order to allow the slide assembly to separately impart a pulling force on each of the two pull wires, one of the two pull wires is directly coupled to the slide assembly whereas the other of the pull wires is indirectly coupled to the slide assembly via a direction reversing element such a pulley or a pin. In other words, in certain embodiments, a means for of coupling the distal ends of the wires to opposite sides of the slide is included in the handle, whereby motion of the slide in one direction will apply tension to one wire while motion of the slide in the other direction will apply tension to the other wire.

In some embodiments, the pull wires comprise a metal. More specifically, in one example, the wires comprise stainless steel. In some embodiments, the wires comprise a drawn 300-series stainless steel wire. In some embodiments, at least one of the pull wires comprise a round wire. In other embodiments, at least one of the pull wires comprise a flat wire which may be a rectangular wire. In one specific embodiment, the wires comprise stainless steel 304V. In one example, wires have a cross-section of about 0.004″×0.015″. In another example, wires have a cross-section of about 0.004″×0.012″.

In some embodiments, in use, the sheath 530 may be inserted within the vasculature of a patient's body and advanced to a target location. The handle 536 may then be manipulated to allow the user to deflect a distal portion of the sheath 530 in the desired direction. In one broad embodiment, a rotational mechanism is provided that allows rotational movement of the knob in one direction to allow longitudinal movement of the slide assembly in one direction within the inner housing (away from a neutral or starting position) to place one of the pull wires in tension. This allows the sheath 530 distal end to be deflected in a first direction. Whereas, in other embodiments, rotation of the knob in a second direction releases the tension in that pull wire and allows the sheath 530 to return to its neutral position. In other embodiments, further rotation of the knob in the second direction allows the slide assembly to translate linearly or longitudinally in the other (opposing) direction within the handle 536 allowing the other of the two pull wires to be placed in tension. This allows the sheath distal end to be deflected in a second direction.

Additionally, as the user rotates the knob clockwise or counterclockwise depending on the desired direction of curvature of the deflectable distal region, the raised thread 551 translates the rotation of the knob to a linear movement (i.e., up and down) of the slider 550. Thus, the raised thread 551 translates a rotation of the knob to a linear movement of the slider 550. In turn the inverted thread 552 translates the linear movement of the slider 550 to a rotational movement of the position ring 542. This is possible since as disclosed above, in some embodiments, the position ring 542 is connected to the inverted thread 55 via a connector tab (not shown). Because of the connector tab, the linear movement of the slider 550 in turn rotates the position ring 542 through the inverted thread. Since the position ring 542 includes bevel gears that are connected to the bevel gears of the position indicator 544, when the position ring 542 is rotated by the inverted thread 552, this in turn rotates the position indicator 544 clockwise or counterclockwise. Thus, in certain embodiments, the rotational movement of the position ring 542 rotates the position indicator 544. Furthermore, in some embodiments, this mechanism allows the position indicator 544 to provide a visual cue of the steerable sheath 530 deflectable distal region curve direction. This allows the user to more conveniently determine the direction of the deflectable distal portion of the steerable sheath 530 and understand the amount of curve of the deflectable distal portion.

In some embodiments, the consideration of the pitch of the raised thread 551 and the inverted thread 552 and the size of the bevel gears on the position ring 542 and the position indicator 544 allows for the position of the position indicator 544 to match with the curve angle of the deflectable distal region the steerable sheath 530. In some embodiments, the diameter of the slider 550 is between 6 mm to 10 mm. In certain embodiments, the diameter of the slider 550 is 8.97 mm. In some embodiments, the slider 550 may travel in a linear direction between 10 mm to 20 mm. In certain embodiments, the slider 550 may travel in a linear direction between 13 mm to 18 mm. In some embodiments, the pitch of the raised thread 551 is between 10 mm to 15 mm. In other embodiments, the pitch of the raised thread 551 is between 12 mm to 13 mm. In certain embodiments, the pitch of the raised thread 551 is 12.7 mm. In certain embodiments, the pitch ratio of the raised thread 551 to the inverted thread 552 is 4:1. In some embodiments, the pitch of the inverted thread 552 is between 45 mm to 55 mm. In certain embodiments, the pitch of the inverted thread 552 is 50.8 mm. In some embodiments, one full pitch translates to a 90 degree turn of the position indicator 554. Thus, in other embodiments, two full pitches translate to a 180 degree turn of the position indicator 554. In some embodiments, the pitch ratio may be adjusted based on the desired application and/or procedure.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A steerable sheath comprising:
an elongate member defining a lumen, the elongate member having a proximal portion and a distal portion terminating in a deflectable distal tip; and
a handle defining a handle lumen having a handle proximal portion and a handle distal portion that is connected to the proximal portion of the elongate member, the handle comprising:
a rotatable knob;
a slider running through a portion of the handle having a raised thread and an inverted thread, a first direction of the raised thread being inverse to a second direction of the inverted thread; and
a position ring located proximate a position indicator, the position ring and the position indicator each having one or more bevel gears connecting the position ring to the position indicator, the position indicator located proximate the knob;
wherein rotation of the knob acts upon the raised thread to cause a linear movement of the slider which acts upon the inverted thread to cause a rotational movement of the position ring.

2. The steerable sheath of claim 1, wherein the one or more bevel gears of the position ring rotate the one or more bevel gears of the position indicator, and wherein the rotational movement of the position ring translates to rotation of the position indicator.

3. The steerable sheath of claim 1, wherein the direction of the position indicator coincides with the curved direction of the deflectable distal tip.

4. The steerable sheath of claim 1, wherein the handle further includes two pull wires connected to the slider, and wherein the pull wires control the curve direction of the deflectable distal tip.

5. The steerable sheath of claim 1, wherein the position ring is connected to the inverted thread by a connector tab.

6. The steerable sheath of claim 1, wherein the knob is rotated by a user depending on the desired direction of curvature of the deflectable distal tip, and wherein the position indicator provides a visual cue to the user indicating the curve direction of the deflectable distal tip of the steerable sheath.

7. The steerable sheath of claim 1, wherein the pitch of the raised thread is higher than the pitch of the inverted thread.

8. The steerable sheath of claim 1, wherein the pitch ratio of the raised thread to the inverted thread is 4:1.

9. The steerable sheath of claim 1, wherein the pitch of the raised thread is between 10 mm to 15 mm.

10. The steerable sheath of claim 1, wherein the pitch of the inverted thread is between 45 mm to 55 mm.

11. The steerable sheath of claim 1, wherein the system further includes a puncture device and an ablation device configured to advance through the steerable sheath.

12. A steerable sheath for transseptal crossing and subsequent therapy, the steerable sheath comprising:
an elongate member defining a lumen, the elongate member having a proximal portion and a distal portion terminating in a deflectable distal tip; and
a handle defining a handle lumen having a handle proximal portion and a handle distal portion that is connected to the proximal portion of the elongate member, the handle comprising:
a knob positioned at the distal portion of the handle;
a slider running through a portion of the handle having a raised thread and an inverted thread, a first direction of the raised thread being inverse to a second direction of the inverted thread; and
a position ring located proximate a position indicator, the position ring and the position indicator each having one or more bevel gears connecting the position ring to the position indicator, the position indicator located proximate the knob;
wherein rotation of the knob acts upon the raised thread to cause a linear movement of the slider which acts upon the inverted thread to cause a rotational movement of position ring;
wherein the one or more bevel gears of the position ring rotate the one or more bevel gears of the position indicator, and wherein the rotational movement of the position ring translates to rotation of the position indicator.

13. The steerable sheath of claim 12, wherein the direction of the position indicator coincides with the curved direction of the deflectable distal tip.

14. The steerable sheath of claim 12, wherein the handle further includes two pull wires connected to the slider, and wherein the pull wires control the curve direction of the deflectable distal tip.

15. The steerable sheath of claim 12, wherein the position ring is connected to the inverted thread by a connector tab.

16. The steerable sheath of claim 12, wherein the knob is rotated by a user depending on the desired direction of curvature of the deflectable distal tip, and wherein the position indicator provides a visual cue to the user indicating the curve direction of the deflectable distal tip of the steerable sheath.

17. The steerable sheath of claim 12, wherein the pitch of the raised thread is higher than the pitch of the inverted thread.

18. The steerable sheath of claim 12, wherein the system further includes a puncture device and a subsequent medical therapy device configured to advance through the steerable sheath.

19. The steerable sheath of claim 12, wherein the system further includes a subsequent medical therapy device configured to advance through the steerable sheath.

20. A method of using a steerable sheath for transseptal crossing and subsequent therapy, the method comprising:
providing an elongate member defining a lumen, the elongate member having a proximal portion and a distal portion terminating in a deflectable distal tip;
advancing a handle defining a handle lumen having a handle proximal portion and a handle distal portion that is connected to the proximal portion of the elongate member, the handle comprising;
a knob positioned at the distal portion of the handle;
a slider running through a portion of the handle having a raised thread and an inverted thread, a first direction of the raised thread being inverse to a second direction of the inverted thread; and
a position ring located proximate a position indicator, the position ring and the position indicator each having one or more bevel gears connecting the position ring to the position indicator, the position indicator located proximate the knob;
wherein rotation of the knob acts upon the raised thread to cause a linear movement of the slider which acts upon the inverted thread to cause a rotation movement position ring.
